(19) United States
(12) Patent Application Publication
(10) Pub. No.: US 2013/0079398 A1
(43) Pub. Date: Mar. 28, 2013

(54) PHARMACEUTICAL COMPOSITION FOR PREVENTING OR TREATING NERVOUS SYSTEM DISORDERS COMPRISING SULFURETIN OR PHARMACEUTICALLY ACCEPTABLE SALT THEREOF

(71) Applicant: Choon Gon Jang, Suwon-si (KR)

(72) Inventor: Choon Gon Jang, Suwon-si (KR)

(21) Appl. No.: 13/637,579

(22) PCT Filed: Sep. 24, 2010
(86) PCT No.: PCT/KR10/06519

§ 371 Date: Dec. 5, 2012

(30) Foreign Application Priority Data
Mar. 26, 2010 (KR) 10-2010-0027560

(51) Int. Cl. A61K 31/343

(52) U.S. Cl. CPC A61K 31/343
USPC 514/470; 549/466

(57) ABSTRACT

The present invention relates to a pharmaceutical composition for preventing or treating nervous system disorders, which contains sulfuretin or a pharmaceutically acceptable salt thereof, and a method of using the composition to prevent or treat nervous system disorders. Moreover, the present invention relates to a functional food composition for alleviating nervous system disorders, which contains sulfuretin or a pharmaceutically acceptable salt thereof. The pharmaceutical composition and the functional food composition can be effectively used to prevent or treat degenerative brain disorders caused by a variety of cerebral nervous system abnormalities in persons, as well as depressive disorder and anxiety.

### TECHNICAL FIELD

The present invention relates to a pharmaceutical composition for preventing or treating nervous system disorders, which comprises sulfuretin or a pharmaceutically acceptable salt thereof, and a method of using the composition to prevent or treat nervous system disorders. Moreover, the present invention relates to a functional food composition for alleviating nervous system disorders, which comprises sulfuretin or a pharmaceutically acceptable salt thereof.

### BACKGROUND ART

The present invention relates to sulfuretin(3′,4′,6-trihydroxyaurone and a pharmaceutically acceptable salt thereof, which have anti-dementia, antioxidant and nerve protective effects, and more particularly to a pharmaceutical composition and a functional food composition, which contain sulfuretin or a pharmaceutically acceptable salt thereof and is used to prevent or treat degenerative brain diseases caused by a variety of cerebral nervous system abnormalities in persons, as well as stress, oxidative damages, aging, depressive disorder and anxiety.

Nerve cells continue to undergo cell death during development and synaptic reconstruction, and nerve cell death caused by stress and cytotoxic drugs is a major cause of brain disease. Among them, oxidative stress is known to have a significant connection with the cause of degenerative brain diseases such as Alzheimer's disease, Parkinson's disease and stroke (Markesbery, Oxidative Stress Hypothesis in Alzheimer's Disease, Free Radical Biology & Medicine, 1997, v. 23, pp. 134-147). Recent studies showed that chronic stress and oxidative stress increase cell death in the hypothalamo-pituitary-adrenocortical axis, hippocampus, striatum, subtantia nigra and frontal cortex areas and reduce neurons and growth factors to cause dementia, Alzheimer's disease, Parkinson's disease, stroke, anxiety and depressive disorder (Yu et al., Alzheimer's Disease and Oxidative Stress, 2006, v. 23, pp.1142-1148; Mirescu et al., Stress and Adult Neurogenesis, Hippocampus, 2006, v. 16, pp. 233-238; Lotharius and Brundin, Pathogenesis of Parkinson's Disease: Dopamine, Vesicles and Alpha-Synuclein, Neuroscience, 2002, v. 3, pp. 932-942; Lee et al., Repression of phospho-JNK and infarct volume in ischemic brain of JIP1-deficient mice, Journal of Neuroscience Research, 2001, v. 74, pp. 326-332; Graham, Hostility and Pain are Related to Inflammation in Older Adults, Brain, Behavior, and Immunity, 2006 v. 20, pp 389-400).

Particularly, free radicals from oxygen are known as a major cause of tissue injury, and oxygen radicals associated with neurotoxicity include hydrogen peroxide (H₂O₂), hydrogen peroxide anions (O₂⁻), hydroxyl radicals (•OH) and the like. Among them, hydrogen peroxide is known as the most important substance as a precursor of a highly reactive free radical and is likely to cause cell death in the central nervous system (McCord, 1985, Free radicals and myocardial ischemia: Overview and outlook, 1988, Free radical biology & medicine, v. 4, pp. 9-14; Rantan et al., Ascorbic acid and focal cerebral ischaemia in a primate model, 1994, Acta neurochirurgica, v. 123, pp. 87-91).

If brain nerve cells undergo oxidative stress, reactive oxygen species (ROS) are triggered to cause cytochrome C release and caspase-3 activation in mitochondria, resulting in cell death. In addition, ROS result in the activation of glutamate, particularly the NMDA receptor, which increases Ca²⁺ ions by the metabotrophic cascade, and the increase in intracellular Ca²⁺ associated with ROS also results in caspase-2 activation causing DNA damage (Annunziato et al., Apoptosis Induced in Neuronal Cells by Oxidative Stress: Role Played by Caspases and Intracellular Calcium Ions, 2003, Toxicology Letter, v. 139, pp. 125-133). It is known that cell death by cerebral ischemic injury results from excitatory neurotoxicity caused by the disruption of homeostasis of Ca²⁺ ions, endoplasmic reticulum dysfunction, mitochondrial dysfunction, and DNA damage caused by oxidative stress (Wei et al., The Antioxidant EPC-K1 Attenuates NO-induced Mitochondrial Dysfunction, Lipid Peroxidation and Apoptosis in Cerebellar Granule Cells, 1999, Toxicology, v. 134, pp. 117-126).

The rate of deaths caused by degenerative brain diseases, and depressive disorder and anxiety by chronic stress is increasing annually worldwide. Currently, a great deal of interest is being focused on the development of drugs for treating degenerative brain diseases, including dementia, Alzheimer's disease, Parkinson's disease and strokes, as well as depressive disorder and anxiety. However, clinically effective drugs for these diseases should be used carefully due to their side effects, and a drug capable of treating or preventing various diseases simultaneously is not yet known.

Meanwhile, antioxidants that eliminate substances harmful to the human body have been successfully used to protect synthetic products or foods from oxidation. Recently, these antioxidants have been studied and developed further as nerve protective drugs for degenerative brain diseases caused by oxidative stress.

Sulfuretin or a pharmaceutically acceptable salt, which is used in the present invention, is the main component of Albizziae julibrissin and was reported to have anticancer, anti rheumatoid arthritis, anti-inflammatory, anti-platelet coagulation and anti-allergic effects (Jeon et al., Anti-platelet Effects of Bioactive Compounds Isolated from the Bark of Rhus verniciflua Stokes, 2006, Journal of Ethnopharmacology, v. 105, pp. 62-69; Choi et al., Sulfuretin, an Antinociceptive and Antiinflammatory Flavonoid from Rhus verniciflua, 2003, Natural Product Sciences, v. 9, pp. 97-101; Jang et al., Flavonoids purified from Rhus Verniciflua Stokes actively Inhibit Cell Growth and Induce Apoptosis in Human Osteosarcoma Cells, 2005, Biochimica et Biophysica Acta, General subjects, v. 1726, pp. 309-316; Jung et al, Antioxidant Activity from the Stem Bark of Albizzia julibrissin, 2003, Archives of Pharmacal Research, v. 26, pp. 458-462). However, it has not yet been reported that sulfuretin or a pharmaceutically acceptable salt thereof is effective against nervous system disorders.

### DISCLOSURE

### Technical Problem

In this regard, the present inventors have found that sulfuretin or a pharmaceutically acceptable salt thereof has the effect of protecting nerve cells to prevent or treat dementia, Alzheimer's disease, Parkinson's disease, stroke, depressive disorder and anxiety, thereby completing the present invention.

### Technical Solution

It is an object of the present invention to provide a pharmaceutical composition for preventing or treating a nervous system disorder, which comprises sulfuretin or a pharmaceutically acceptable salt thereof.

Another object of the present invention is to provide a functional food composition alleviating nervous system disorders, which comprises sulfuretin or a pharmaceutically acceptable salt thereof.

Still another object of the present invention is to provide a method of preventing or treating a nervous system disorder by administering a pharmaceutical composition comprising sulfuretin or a pharmaceutically acceptable salt thereof to a subject having or being at risk of developing the nervous system disorder.

### Advantageous Effects

Sulfuretin or a pharmaceutically acceptable salt thereof according to the present invention can be used as a drug and a functional food, which have the effect of preventing or treating degenerative brain disease caused by a variety of cerebral nervous system abnormalities in persons, as well as treating depressive disorder and anxiety.

### DESCRIPTION OF DRAWINGS

FIG. 1 shows the chemical structure of sulfuretin.

FIG. 2 shows the free radical elimination ability of sulfuretin in a test for free radical elimination ability (antioxidant effects).

FIG. 3 shows the nerve cell protective effects of sulfuretin in cell death induced by hydrogen peroxide, beta-amyloid, 6-hydroxydopamine, corticosterone and SNP in SH-SY5Y and PC12 cells.

FIG. 4 shows the effect of sulfuretin on the inhibition of LDH secretion induced by hydrogen peroxide and beta-amyloid in SH-SY5Y cells.

FIG. 5 shows the effect of sulfuretin on the inhibition of ROS production induced by hydrogen peroxide in SH-SY5Y cells.

FIG. 6 shows the effect of sulfuretin on the inhibition of intracellular [Ca²⁺] influx induced by hydrogen peroxide in SH-SY5Y cells.

FIG. 7 shows the effect of sulfuretin on the inhibition of mitochondrial membrane potential damage induced by hydrogen peroxide in SH-SY5Y cells.

FIG. 8 shows the effect of sulfuretin on the expression of cell death-related proteins induced by hydrogen peroxide in SH-SY5Y cells.

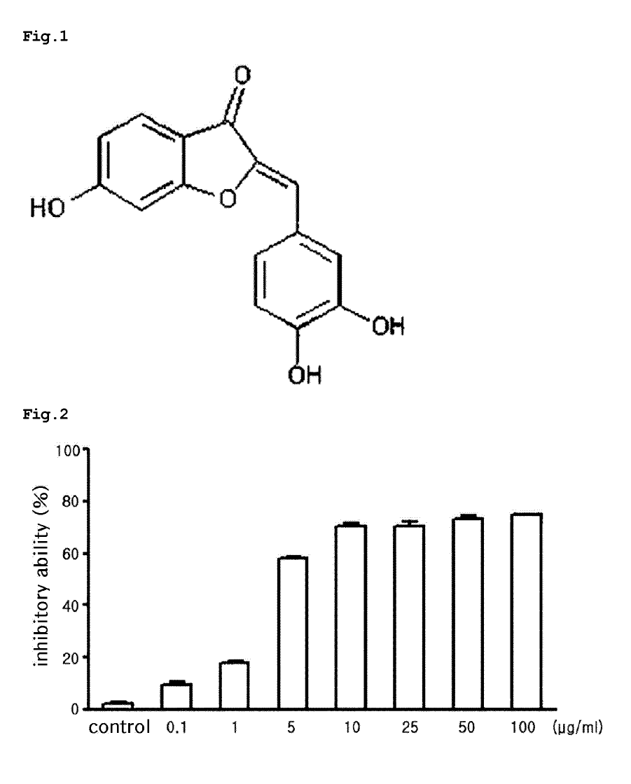

### BEST MODE

In one aspect, the present invention is directed to a pharmaceutical composition for preventing or treating nervous system disorders, which contains sulfuretin or a pharmaceutically acceptable salt thereof.

In another aspect, the present invention is directed to a method of preventing or treating a nervous system disorder by administering a pharmaceutical composition comprising sulfuretin or a pharmaceutically acceptable salt thereof to a subject having or being at risk of developing the nervous system disorder.

As used herein, the term “sulfuretin” refers to a compound of the following formula 1:

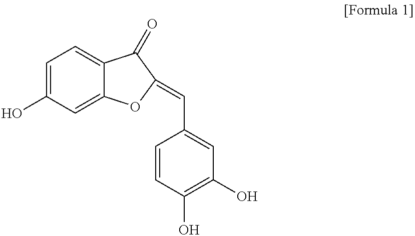 [SMILES: O=C1/C(=C/c2ccc(O)c(O)c2)Oc2cc(O)ccc21]

Sulfuretin is a flavonoid naturally obtained from plants, including Albizziae julibrissin and Rhus Verniciflua, or may be artificially synthesized by a method known in the art.

As used herein, the term “pharmaceutically acceptable salt” refers to a salt prepared by a conventional method and is known to one skilled in the art. Pharmaceutically acceptable salts of sulfuretin include those derived from the following pharmacologically or physiologically acceptable inorganic and organic acids : hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulphonic and benzenesulfonic acids, but not being limited therein. Salts may also be derived from the following pharmacologically or physiologically acceptable inorganic and organic bases: alkali metal (e.g. sodium), alkaline earth metal (e.g. magnesium), and ammonium salts, but not being limited therein.

As used herein, the term “nervous system disorder” is meant to include dementia, Alzheimer's disease, Parkinson's disease, stress, oxidative conditions, aging, stroke, depressive disorders and anxiety.

As used herein, the term “preventing” refers to all actions that inhibit or delay the disorders by the administration of a composition comprising sulfuretin or a pharmaceutically acceptable salt thereof according to the present invention.

As used herein, the term “treating” refers to all actions that restore or beneficially change the disorders by the administration of a composition comprising sulfuretin or a pharmaceutically acceptable salt thereof according to the present invention.

In one specific embodiment of the present invention, the present inventors found that sulfuretin has effects on the prevention and treatment of nervous system disorders. More specifically, it could be seen that the ability of sulfuretin to eliminate free radicals increases in a dose-dependent manner, suggesting that it has an antioxidant effect (FIG. 2).

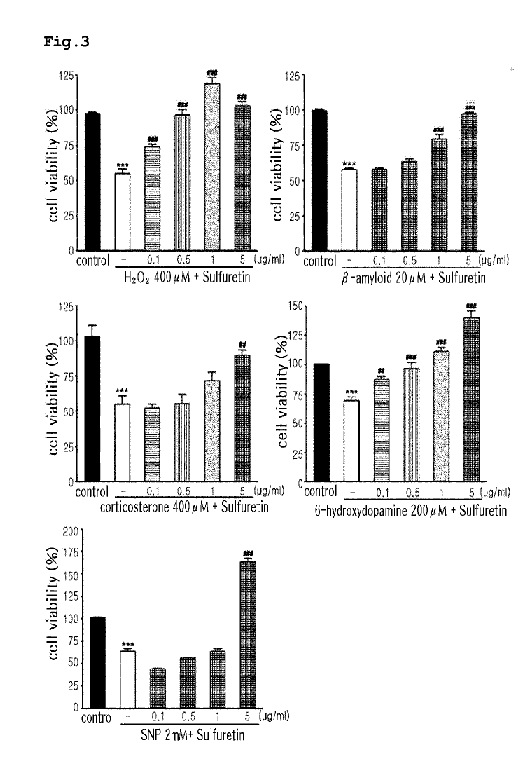

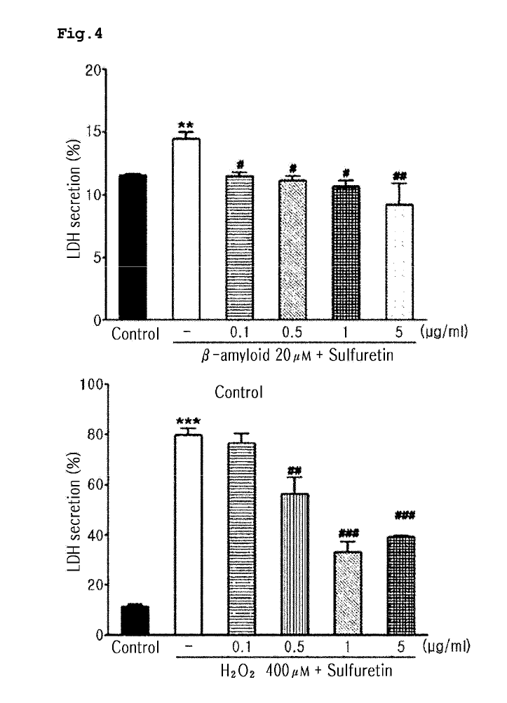

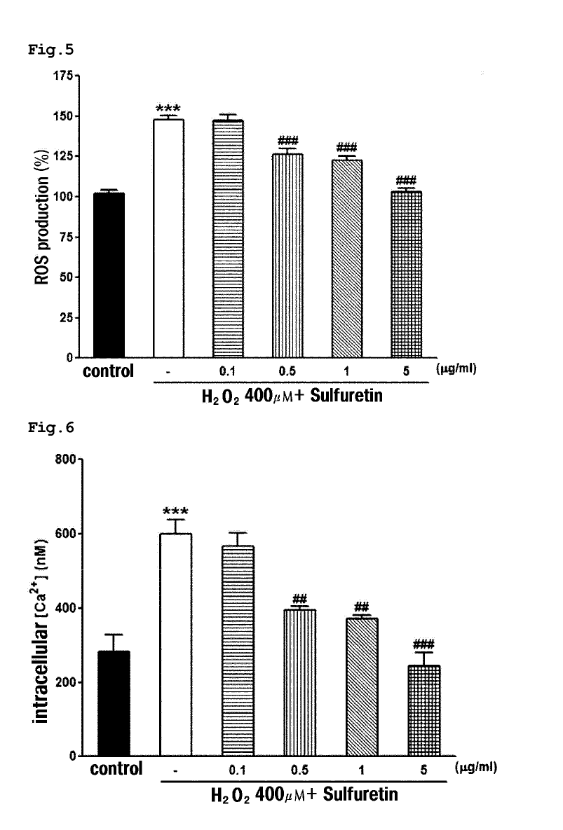

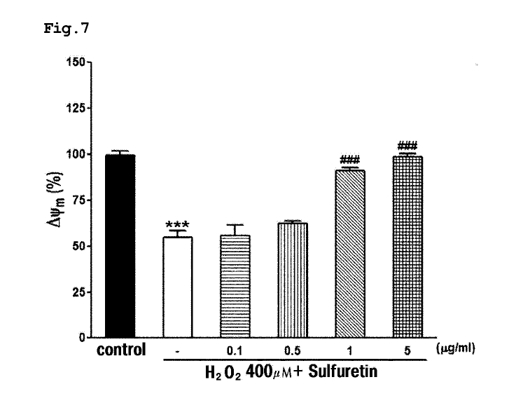

Also, it could be seen that sulfuretin showed nerve cell protective effects in cell death induced by hydrogen peroxide, beta-amyloid, 6-hydroxydopamine, corticosterone and SNP in an MTT (cell viability) test in SH-SY5Y and PC12 cells (FIG. 3). Further, it could be seen that sulfuretin inhibited LDH secretion in a test for measurement of LDH secretion induced by hydrogen peroxide and beta-amyloid in SH-SY5Y cells, suggesting that it has no cytotoxicity (FIG. 4). Moreover, it could be seen that sulfuretin inhibited ROS production in a test for inhibition of ROS production induced by hydrogen peroxide in SH-SY5Y cells (FIG. 5). In addition, it could be seen that sulfuretin inhibited intracellular [Ca²⁺] influx induced by hydrogen peroxide in SH-SY5Y cells (FIG. 6). Furthermore, it could be seen that sulfuretin inhibited mitochondrial membrane potential damage induced by hydrogen peroxide in SH-SY5Y cells, suggesting that it recovers mitochondrial membrane potential (FIG. 7).

Thus, it can be seen that sulfuretin or a pharmaceutically acceptable salt thereof is effective in preventing or treating nervous system disorders with antioxidant and nerve cell protective effects.

The composition comprising sulfuretin or a pharmaceutically acceptable salt thereof according to the present invention may further comprise an appropriate carrier, excipient or diluent according to a conventional method.

As used herein, the term “carrier” refers to a carrier, excipient or diluent that does not significantly irritate an organism and does not reduce the biological activity and properties of the compound administered.

Examples of carriers, excipients and diluents that may be included in the composition of the present invention include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

The composition comprising sulfuretin or a pharmaceutically acceptable salt thereof according to the present invention can be formulated according to conventional methods for oral dosage forms such as powders, granules, tablets, capsules, suspensions, emulsions, syrups, or aerosols; external dosage forms; suppositories; or sterile injection solution.

The composition of the present invention can be formulated using conventional diluents or excipients, including fillers, extenders, binders, wetting agents, disintegrants, and surfactants.

Solid formulations for oral administration include tablets, pills, powders, granules, capsules, etc. These solid formulations may be prepared by mixing at least one compound with one or more excipients, for example, starch, calcium carbonate, sucrose, lactose, gelatin, etc. In addition to regular excipients, lubricants such as magnesium stearate and talc may be used.

In addition, liquid formulations for oral administration include suspensions, solutions, emulsions and syrups, etc. In addition to water commonly used as a simple diluent and liquid paraffin, various excipients, for example, wetting agents, sweetening agents, flavors, preservatives, etc. may be included. Formulations for parenteral administration include sterilized aqueous solutions, non-aqueous solvents, suspending agents, emulsions, freeze-drying agents, suppositories, etc. Propylene glycol, polyethylene glycol, vegetable oils such as olive oil, injectable esters such as ethyl oleate, etc. may be used as non-aqueous solutions and suspending agents. Suppositories may include witepsol, macrogol, tween 61, cacao butter, laurin butter, glycerinated gelatin, etc.

The pharmaceutical composition for preventing or treating nervous system disorders according to the present invention may be administered in a pharmaceutically effective amount. As used herein, the term “pharmaceutically effective amount” refers to an amount sufficient to treat diseases, at a reasonable benefit/risk ratio applicable to any medical treatment. The effective dosage level of the composition may be determined depending on the subject's type, the disease severity, the subject's age and sex, the type of infected virus, the activity of the drug, sensitivity to the drug, the time of administration, the route of administration, excretion rate, the duration of treatment, drugs used in combination with the composition, and other factors known in the medical field.

The dose of sulfuretin or a pharmaceutically acceptable salt thereof according to the present invention may depend on the age, sex, and weight of a patient, but the extract may be administered in an amount of generally 0.01 to 500 mg/kg, and preferably 0.1 to 100 mg/kg once or several times a day. Also, the dose of sulfuretin or a pharmaceutically acceptable salt thereof may be increased or decreased according to an administration route, severity of a disease, sex, weight, age, etc. Therefore, the dose does not limit the scope of the present invention in any way.

The pharmaceutical composition according to the present invention may be administered to mammals such as a rat, a mouse, a domestic animal, a human, etc. through various routes. The administration of the composition may be carried out through all possible methods, for example, oral administration, rectal administration, intravenous injection, intramuscular injection, subcutaneous injection, intra-endometrial injection, intracerebroventricular injection.

In another aspect, the present invention provides a health functional food for preventing nervous system-related psychiatric disorders, which comprises sulfuretin or a pharmaceutically acceptable salt thereof and food-acceptable additives.

The inventive composition comprising sulfuretin or a pharmaceutically acceptable salt thereof may be used in drugs, foods and beverages for preventing nervous system-related psychiatric disorders. Foods to which the compound of the present invention can be added include various foods, for example, beverages, gums, teas, vitamin complexes, health supplement foods, etc., and can be used in the form of pills, powders, granules, infusions, tablets, capsules, or drinks.

The amount of the compound of the present invention in the food or beverage product may generally range from 0.01 to 15 wt % based on the total weight of the food for a health food composition, and from 0.02 to 10 g, preferably 0.3 to 1 g, per 100 ml for a health beverage composition.

Besides including the above compound as an active ingredient in the percentage indicated above, the health beverage composition does not include any particular limitations on the liquid component and can include additional ingredients, such as various flavorings or natural carbohydrates, etc., as is common in typical beverages. Examples of natural carbohydrates include common sugars, including monosaccharides, such as glucose, fructose, etc., disaccharides, such as maltose, sucrose, etc., and polysaccharides, such as dextrin, cyclodextrin, etc., as well as sugar alcohols, such as xylitol, sorbitol, erythritol, etc. In addition, other flavorings can advantageously be used, including natural flavorings (thaumatin, stevia extracts, such as rebaudioside A, glycyrrhizin, etc.) and synthetic flavorings (saccharin, aspartame, etc.). The content of the natural carbohydrates is generally about 1 to 20 g, preferably about 5 to 12 g, per 100 ml of the composition of the present invention.

In addition to the above, the composition of the present invention may contain various nutrients, vitamins, minerals (electrolyte), flavoring agents such as synthetic flavoring agents and natural flavoring agents, coloring agents and improving agents (cheese, chocolate, etc.), pectic acid and salts thereof, alginic acid and salts thereof, organic acids, protective colloidal thickening agents, pH controlling agents, stabilizing agents, preservatives, glycerin, alcohol, carbonizing agents as used in carbonated beverages, etc. Moreover, the compositions of the present invention may contain fruits, as used in preparing natural fruit juices and fruit juice beverages and vegetable beverages. These components can be used independently or in combination. Although the percentage of the additive is not of great importance, it is generally selected from a range of 0 to about 20 parts by weight per 100 parts by weight of the composition of the present invention.

The functional food composition of the present invention may be in the form of pills, powders, granules, infusions, tablets, capsules or liquids, and examples of foods to which the composition of the present invention include various foods, for example, beverages, gums, teas, vitamin complexes, health supplement foods, etc.

Besides the composition having effects against nerve system disorders, which contains sulfuretin or a pharmaceutically acceptable salt thereof as an essential ingredient, the functional food composition of the present invention may include other ingredients without particular limitations, and may include various herbal medicinal extracts, food additives, or natural carbohydrates in the same manner of conventional foods.

The functional food composition may further include one or more herbal medicinal extracts selected from the group consisting of Japanese apricot, Gastrodia elata Blume, Schizandra chinensis, Glycyrrhiza uralensis, Cassia obtusifolia, Acorus graminens, Sepia bone, Polygalae radix, Astragalus membranaceus, Semen ziziphi spinosae, Bupleurum falcatum, Atractyloides chinensis, Angelica gigas. Lycium barbarum, and Poria cocos.

In addition, a food supplement additive may be further included, and food additives include a fragrance agent, a flavoring agent, a coloring agent, a filler, a stabilizer or the like, which is conventionally known in the art.

Examples of the natural carbohydrate include conventional sugar, such as monosaccharide (e.g., glucose, fructose, etc.);

disaccharide (e.g., maltose, sucrose, etc.); polysaccharide (e.g., dextrin, cyclodextrin, etc.); and sugar alcohol such as xylitol, sorbitol, erythritol, etc. Also, as a fragrance agent, a natural fragrance agent such as thaumatin, a stevia extract (e.g., rebaudioside A, glycyrrhizin, etc.) and a synthetic fragrance agent such as saccharine, aspartame, etc. may advantageously be used.

In addition, the functional food composition of the present invention may contain various nutrients, vitamins, minerals (electrolytes), a flavor agent (such as a synthetic flavor agent, a natural flavor agent, etc.), a coloring agent, an extender (cheese, chocolate, etc.), pectic acid and its salt, alginic acid and its salt, organic acid, a protective colloid thickener, a PH adjuster, a stabilizer, a preservative, glycerin, alcohol, a carbonating agent used for a carbonated drink, etc. In addition, the functional food composition of the present invention may contain flesh that may be used for preparing natural fruit juice, fruit juice drinks, and vegetable drinks. Such components may be used independently or in combination.

### MODE FOR INVENTION

Hereinafter, the present invention will be described in detail with reference to examples. It is to be understood, however, that these examples are for illustrative purposes only and are not intended to limit the scope of the present invention.

### EXAMPLE 1

### Drugs and Reagents

Sulfuretin used in the present invention was purchased from Extrasynthese (France); amyloid-beat 25-35 frayment, Corticosterone, 2′,7′-dichlorofluoroscein diacetate (DCFH-DA), 2,2-diphenyl-1-picrylhydrazyl (DPPH), dimethylsulfoxide (DMSO), 6-hydroxydopamine, 30% hydrogen peroxide (H₂O₂), Fura-2-AM, Rhodamine-123, 3-(4,5-dimethylthiazol-2-yl)-2,5-diphenyltetrazolium bromide (MTT), sodium nitroprusside (SNP), and anti-(3-actin were purchased from Sigma-Aldrich Chemistry Co.; anti-PARP (poly ADP ribosepolymerase), anti-capase-3, anti-phospho-p38, and anti-phospho-JNK antibodies were purchased from Cellsignaling (USA); and an LDH cytotoxicity assay kit was purchased from Takara (Japan). In addition, reagents used in the experiment had the highest quality.

### EXAMPLE 2

### Preparation of SH-SY5Y Nerve Cell Line

Human neuroblastoma SH-SY5Y cells were cultured in DMEM medium (dulbecco's modified eagle's medium) (Hyclone, Thermo, USA) containing 10% fetal bovine serum (FBS) and antibiotics (Gibco-BRL, USA). The incubator was maintained at a temperature of 37° C., and a mixed gas of 95% air and 5% CO₂ was continuously supplied thereto to provide suitable conditions for cell culture. The cells were cultured in a 6-well plate or a 96-well plate at each of densities of 1×10⁶ and 2×10⁴ cells/well 24 hours before the experiment. The concentrations of hydrogen peroxide, beta-amyloid and 6-hydroxydopamine were determined to be 400 μM, 20 μM and 200 μM, respectively, after evaluation of cell viability. Sulfuretin was used in ethanol and used at a final concentration of 0.1% or less.

### EXAMPLE 3

### Preparation of PC12 Nerve Cell Line

PC12 cells were cultured in DMEM medium (Hyclone, Thermo, USA) containing 10% FBS, 5% horse serum and antibiotics (Gibco-BRL, USA). The incubator was maintained at a temperature of 37° C., and a mixed gas of 95% air and 5% CO₂ was continuously supplied thereto to provide suitable conditions for cell culture. The cells were cultured in a 96-well plate at a density of 2×10⁴ cells/well 24 hours before the experiment. The concentration of corticosterone was determined to be 400 μM after evaluation of cell viability. Sulfuretin was used in ethanol and used at a final concentration of 0.1% or less.

### EXAMPLE 4

### Test for Free Radical Scavenging Ability (Antioxidant Effect) of Sulfuretin

2,2-diphenyl-1-picrylhydrazyl (DPPH) was dissolved in 99.5% ethanol to a concentration of 0.1 mM. Sulfuretin was dissolved and diluted in ethanol to concentrations of 0.1, 1, 5, 10, 25, 50 and 100 μg/ml. 10 μl of the sample was added to 90 μl of DPPH and admixed several times with a pipette, and the mixture was incubated at room temperature for 30 minutes, and the absorbance at 517 nm was measured.

The measured absorbance was substituted into the following equation to determine inhibition (%):

Inhibition (%)=[(O.D. of control−O.D. of test group)/O.D. of control]×100

The results of the test are shown in FIG. 2.

The free radical scavenging ability of sulfuretin increased in a dose-dependent manner, and sulfuretin showed free radical scavenging abilities of 10, 18, 58, 70, 71, 73 and 75% or more at doses of 0.1, 1, 5, 10, 25, 50 and 100 μg/ml, respectively.

### EXAMPLE 5

### MTT (Cell Viability) Test for Sulfuretin

To measure cell viability, an MTT reduction assay was used. An MTT solution was added to each well of the 96-well plate (in which the cells have been cultured) to a final concentration of 0.5 mg/l. The plate was incubated in an incubator for 2 hours, and the medium and the MTT solution were removed, after which DMSO was added thereto and stirred. When DMSO was completely dissolved, the UV absorbance at 540 nm was measured using a microplate reader (Molecular device, USA).

The measured absorbance was substituted into the following equation to calculate cell viability:

Cell viability (%)=[(O.D. of control−O.D. of test group)/O.D. of control]×100

The results of the test are shown in FIG. 3.

As can be seen in FIG. 3, treatment with hydrogen peroxide showed a cell viability of 55% or more, and sulfuretin showed cell viabilities of 74%, 97%, 119% and 103% or more at doses of 0.1, 0.5, 1 and 5 μg/ml, respectively.

Treatment with beta-amyloid showed a cell viability of 58% or more, and sulfuretin showed cell viabilities of 58%, 64%, 80% and 97% or more at a doses of 0.1, 0.5, 1 and 5 μg/ml, respectively.

Treatment with corticosterone showed a cell viability of 55% or more, and sulfuretin showed cell viabilities of 52%, 55%, 72% and 90% or more at doses of 0.1, 0.5, 1 and 5 μg/ml, respectively.

Treatment with 6-hydroxydopamine showed a cell viability of 69% or more, and sulfuretin showed cell viabilities of 87%, 97%, 112% and 140% or more at doses of 0.1, 0.5, 1 and 5 μg/ml, respectively.

Treatment with SNP showed a cell viability of 63% or more, and sulfuretin showed cell viabilities of 44%, 56%, 64%, and 163% or more at doses of 0.1, 0.5, 1 and 5 μg/ml, respectively.

### EXAMPLE 6

### Test for LDH Secretion (Cytotoxicity) of Sulfuretin

To measure LDH secretion (cytotoxicity), an LDH cytotoxicity assay kit was used. 50 μl of the medium was taken from each well of the 96-well plate (in which the cells have been cultured) and dispensed into each well of a fresh 96-well plate, and 50 μl of a reaction reagent was added to each well, after which the plate was shielded from light. The plate was incubated in an incubator for 30 minutes, and the UV absorbance at 490 nm was measured using a microplate reader (Molecular device, USA).

The measured absorbance was substituted into the following equation to calculate LDH secretion:

LDH secretion (%)=(LDH secretion O.D. in medium/total LDH secretion O.D.)×100

The results of the test are shown in FIG. 4.

As can be seen in FIG. 4, in the case of hydrogen peroxide, a normal control group showed LDH secretion of 11% or less, and treatment with hydrogen peroxide showed LDH secretion of 80% or more, and sulfuretin showed LDH secretion of 77%, 56%, 33% and 39% or less at doses of 0.1, 0.5, 1 and 5 μg/ml, respectively.

In the case of beta-amyloid, a normal control group showed LDH secretion of 12% or less, and treatment with beta-amyloid showed an LDH secretion of 15% or more, and sulfuretin showed

LDH secretions of 12%, 11%, 11% and 9% or less at doses of 0.1, 0.5, 1 and 5 μg/ml, respectively.

### EXAMPLE 7

### Test for Effect of Sulfuretin on Inhibition of ROS Production

In order to measure ROS elimination activity, cells were labeled with DCFH-DA fluorescence, and quantitative analysis was performed using a fluorescence meter.

Specifically, the 6-well plate in which the cells have been cultured was pretreated with sulfuretin for 2 hours, and then reacted with hydrogen peroxide for 30 minutes to induce ROS. Then, 10 μl of 10 mM DCFH-DA was added to the plate and allowed to react with the cells for 30 minutes. Herein, DCFH-DA in the incubator was covered with a foil such that it would not be exposed to light. The cells that reacted with the DCFH-DA fluorescent substance were detached using cold PBS and centrifuged at 400 g for 3 minutes. The centrifugation process was repeated twice to wash the fluorescent substance. The washed cells were briefly stirred with 1 ml of PBS, and 100 μl was dispensed into each of a fresh 96-well plate. Then, fluorescence was measured at an excitation wavelength of 488 nm and an emission wavelength of 515 nm using a fluorescence meter (Perkinelmer, USA).

The measured value was substituted into the following equation to calculate ROS production:

ROS production (%)=[(O.D. of control−O.D. of test group)/O.D. of control]×100

The results of the test are shown in FIG. 5.

As can be seen in FIG. 5, a control group showed an ROS production of 102% or less, and treatment with hydrogen peroxide showed an ROS production of 148% or more, and sulfuretin showed ROS productions of 147%, 126%, 123% and 103% or less at doses of 0.1, 0.5, 1 and 5 ug/me, respectively.

### EXAMPLE 8

### Test for Effect of Sulfuretin on Inhibition of Intracellular Influx of [Ca²⁺]

In order to measure inhibitory effects on intracellular [Ca²⁺] influx caused by oxidative stress, cells were labeled with Fura-2-AM fluorescence, and quantitative analysis was carried out using a fluorescence meter. Specifically, 10 μl of 5 μM Fura-2-AM was added to each well of the 6-well plate (in which the cells have been cultured) and then allowed to react for 30 minutes. Herein, Fura-2-AM in the incubator was covered with a silver foil such that it would not be exposed to light. After completion of the incubation, the plate was pretreated with sulfuretin for 30 minutes, and then reacted with hydrogen peroxide for 30 minutes to induce the intracellular influx of [Ca²⁺]. Then, the cells that reacted with the Fura-2-AM fluorescent substance were washed twice with cold HEPES and detached with HEPES. Then, the cells were centrifuged at 400 g for 3 minutes. The supernatant was removed carefully, stirred weakly with 0.5 ml of HEPES buffer, and then 150 μl was dispensed into each well of a fresh 96-well plate. The fluorescence of the sample dispensed into the fresh 96-well plate was measured at an excitation wavelength of 540 nm or 580 nm and an emission wavelength of 520 nm using a fluorescence meter (Perkinelmer, USA).

The measured value was substituted into the following equation to calculate intracellular [Ca²⁺]nM:

Intracellular [Ca²⁺]nM=224 nM×excitation 540/580

The results of the test are shown in FIG. 6.

As can be seen in FIG. 6, a normal control group showed an intracellular [Cah influx of 281 nM or less, and treatment with hydrogen peroxide showed an intracellular [Ca²⁺] influx of 600 nM or more, and sulfuretin showed intracellular [Ca²⁺] influxes of 566 nM, 395 nM, 371 nM and 243 nM or less at doses of 0.1, 0.5, 1 and 5 μg/ml, respectively.

### EXAMPLE 9

### Test for Effect of Sulfuretin on Inhibition of Mitochondrial Membrane Potential (MMP, ΔΨm) Damage

In order to measure the inhibition of MMP damage, cells were labeled with rhodamine-123 fluorescence, and quantitative analysis was performed using a fluorescence meter. Specifically, the 6-well plate in which the cells have been cultured was pretreated with sulfuretin for 30 minutes and then reacted with hydrogen peroxide for 30 minutes to induce MMP damage. Then, 10 μl of 10 μM rhodamine-123 was added to each well of the plate and allowed to react for 30 minutes. Herein, rhodamine-123 in the incubator was covered with a silver foil such that it would not be exposed to light. The cells that reacted with the rhodamine-123 fluorescent substance were detached with cold PBS and centrifuged at 400 g for 3 minutes. The centrifugation process was repeated twice to wash the fluorescent substance. The washed cells were stirred briefly with 0.5 ml of PBS, and 100 μl was dispensed into each well of a fresh 96-well plate. The fluorescence of the cells was measured at an excitation wavelength of 480 nm and an emission wavelength of 530 nm using a fluorescence meter (Perkinelmer, USA).

The measured fluorescence was substituted into the following equation to calculate

ΔΨm(%)=[(O.D. of control−O.D. of test group)/O.D. of control]×100

The results of the test are shown in FIG. 7.

As can be seen in FIG. 7, a normal control group showed an MMP of 99% or less, and treatment with hydrogen peroxide showed an MMP of 55% or less, and sulfuretin showed MMPs of 56%, 62%, 91% and 98% or more at doses of 0.1, 0.5, 1 and 5 μg/ml, respectively.

### EXAMPLE 10

### Test for Effect of Sulfuretin on Expression of Brain Disease-related Proteins

In order to identify the effect of sulfuretin on the expression of proteins related to dementia, Alzheimer's disease, Parkinson's disease and strokes, a Western blot test was carried out.

The 6-well plate in which the cells have been cultured was pretreated with sulfuretin for 2 hours, and then treated with hydrogen peroxide for 24 hours and incubated in an incubator for 24 hours. After 24 hours, the supernatant of each well was collected and centrifuged at 400 g for 3 minutes, and the cells were washed with cold PBS and lysed with 100 μl of T-PER lysis buffer (Thermo, USA) for 30 minutes. The lysates were centrifuged at 10000 g at 4° C. for 15 minutes, and the supernatants were stored at 70° C. until use as test samples. Proteins were quantitatively analyzed using a BCA assay kit (Thermo, USA) and separated within 8.5-12% SDS gel and then transferred to PVDF membranes. To examine the expression of the proteins, the development of fluorescence was analyzed by enhanced chemiluminescence (ECL). Specifically, the membrane was blocked with 5% skimmed non-fat milk for 1 hour and labeled with the primary antibodies, PARP, caspase-3, phospho-p38, phospho-JNK and β-actin at 4° C. overnight. Then, the membrane was washed three times with Tris-tween buffered saline (TTBS), and then labeled with horseradish peroxidase (HRP)-conjugated anti-rabbit and anti-mouse secondary antibodies at room temperature for 1 hour. Then, the membrane was washed five times with TTBS for 10 minutes. The washed film was developed by ECL using x-ray films, and concentrations were quantitatively analyzed using quantitative analysis program (Fujifilm, Japan).

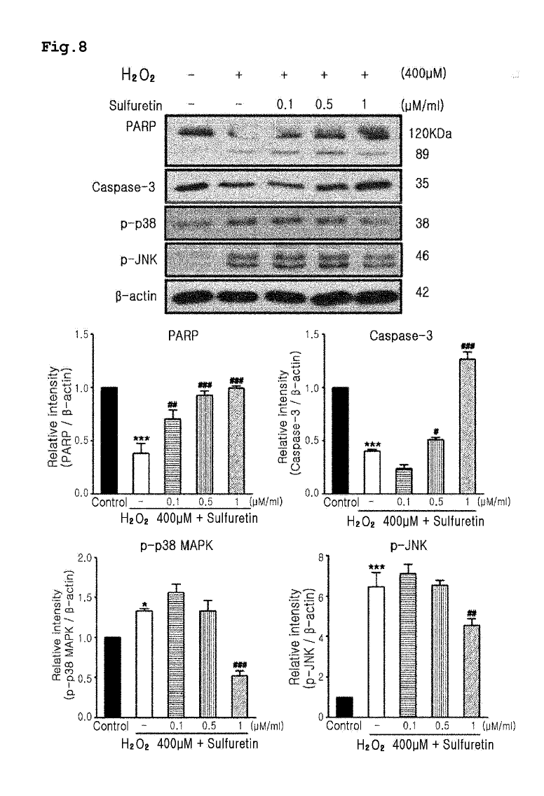

The results of the test are shown in FIG. 8.

As can be seen in FIG. 8, in the expression of PARP protein, a normal control group showed a PARP expression of 100% or more, and treatment with hydrogen peroxide showed a PARP expression of 38% or less, and sulfuretin showed PARP expressions of 70%, 92% and 99% or more at doses of 0.1, 0.5 and 1 μg/ml, respectively.

In the expression of caspase-3 protein, a normal control group showed a caspase-3 expression of 100% or more, and treatment with hydrogen peroxide showed a caspase-3 expression of 40% or less, and sulfuretin showed caspase-3 expressions of 24%, 51% and 127% or more at doses of 0.1, 0.5 and 1 μg/ml, respectively.

In the expression of phospho-p38 protein, a normal control group showed a phospho-p38 expression of 100% or more, and treatment with hydrogen peroxide showed a phospho-p38 expression of 133% or more, and sulfuretin showed phospho-p38 expressions of 157%, 133% and 53% or less at doses of 0.1, 0.5 and 1 μg/ml, respectively.

In the expression of phospho-JNK protein, a normal control group showed a phospho-JNK expression of 100% or more, and treatment with hydrogen peroxide showed a phospho-JNK expression of 173% or more, and sulfuretin showed phospho-JNK expressions of 261%, 204% and 120% or less at doses of 0.1, 0.5 and 1 μg/ml, respectively.

### EXAMPLE 11

### Statistical Processing

All data was statistically processed using one way analysis of variance (ANOVA), and the measurements of significance were performed using Newman-Keuls test (p<0.05).

1. A pharmaceutical composition for preventing or treating a nervous system disorder, comprising sulfuretin or a pharmaceutically acceptable salt thereof.

2. The pharmaceutical composition of claim 1, wherein the nervous system disorder is selected from the group consisting of dementia, Alzheimer's disease, Parkinson's disease, stress, oxidative condition, aging, stroke, depressive disorders and anxiety.

3. The pharmaceutical composition of claim 1, wherein the composition further comprises a pharmaceutically acceptable carrier.

4. A functional food composition for alleviating a nervous system disorder, comprising sulfuretin or a pharmaceutically acceptable salt thereof

5. The functional food composition of claim 4, wherein the composition is in the form of pills, powders, granules, infusions, tablets, capsules or drinks

6. A method of preventing or treating a nervous system disorder by administering the composition of claim 1 to a subject having or being at risk of developing the nervous system disorder.

7. The method of claim 6, wherein the nervous system disorder is selected from the group consisting of dementia, Alzheimer's disease, Parkinson's disease, stress, oxidative condition, aging, stroke, depressive disorder and anxiety.

8. The method of claim 6, wherein the composition further comprises a pharmaceutically acceptable carrier.
